SEQUENCE LISTING (8 sequences)

SEQ ID NO: 1 - protein, 244 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLT

SEQ ID NO: 2 - protein, 323 aa, Homo sapiens
MARGPGLAPPPLRLPLLLLVLAAVTGHTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLTAGLIAVIVVVVVALVAGMAVLVITNRRKSGKYKKVEIKELGELRKEPSL

SEQ ID NO: 3 - protein, 115 aa, Homo sapiens
QDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRC

SEQ ID NO: 4 - protein, 129 aa, Homo sapiens
DELVRTHHILIDLRHRPTAGAFNHSDLDAELRRLFRERYRLHPKFVAAVHYEQPTIQIELRQNTSQKAAGEVDIGDAAYYFERDIKGESLFQGRGGLDLRVRGEPLQVERTLIYYLDEIPPKFSMKRLT

SEQ ID NO: 5 - protein, 119 aa, Homo sapiens
QVQLVQSGAEVKKPGASVKVSCKASGFTFSSSYISWLRQAPGQRLEWIAWIYAGTGGTSYNQKFTGKATLTVDTSASTAYMELSSLRSEDTAVYYCARHNPRYYAMDYWGQGTTVTVSS

SEQ ID NO: 6 - DNA, 357 bp, Homo sapiens
  source 1..357: mol_type = genomic DNA
caagtgcagctcgtccagtctggagctgaagtcaaaaagcctggggcttcagtgaaagtctcctgcaaggcttctggcttcaccttcagcagtagctatatcagttggttgaggcaggcccctggacagagacttgagtggattgcatggatttatgctggaactggcggaactagctataatcagaagttcacaggcaaggccacactgactgtagacacatccgccagcacagcctacatggaactcagcagcctgagatctgaggacactgccgtctattactgtgcaagacataaccctcgttactatgctatggactactggggccaaggaaccacagtcaccgtctcctca

SEQ ID NO: 7 - protein, 108 aa, Homo sapiens
DTQMTQSPSSLSASVGDRVTITCITSTDIDDDMNWYQQKPGKAPKLLISEGNTLRPGVPSRFSGSGYGTDFTFTISSLQPEDIATYYCLQSDNLPYTFGGGTKVEIKR

SEQ ID NO: 8 - DNA, 324 bp, Homo sapiens
  source 1..324: mol_type = genomic DNA
gacacccagatgacccagtctccaagctccctgtccgccagcgtgggagatagagtcaccatcacatgcatcaccagcactgatattgatgatgatatgaactggtaccagcagaagccagggaaagctcctaagctcctgatttcagaaggcaatactctgcgccctggagtcccatcccgattctccggcagtggctatggaacagattttacctttacaattagctccctgcagccagaagatattgcaacctactactgtttgcaaagtgataacctgccctacaccttcggaggggggaccaaagtcgaaatcaaacgg

(19) United States
(12) Patent Application Publication
(10) Pub. No.: US 2026/0184818 A1
(43) Pub. Date: Jul. 2, 2026

(54) ANTI-IDIOTYPE ANTIBODIES AND USES THEREOF

(71) Applicant: MEDITERRANEA THERANOSTIC S.R.L., Lanciano (IT)

(72) Inventor: Saverio ALBERTI, Lanciano (IT)

(73) Assignee: MEDITERRANEA THERANOSTIC S.R.L., Lanciano (IT)

(21) Appl. No.: 18/865,497

(22) PCT Filed: May 29, 2023
(86) PCT No.: PCT/EP2023/064338

§ 371 Date: Nov. 13, 2024

(30) Foreign Application Priority Data
May 30, 2022 (IT) 102022000011402

(51) Int. Cl. C07K 16/42; A61K 40/11; A61K 40/31; A61K 40/42

(52) U.S. Cl. CPC C07K 16/4266; A61K 40/11; A61K 40/31; A61K 40/4202

(57) ABSTRACT

Chimeric antigen receptors (CARs) that are expressible by cells of the immune system such as T cells or NK cells (CAR-T and CAR-NK). These CARs comprise a fragment of scFv antibody directed against a specific antigen of tumor tissues, the tumor activated Trop-2 antigen wherein said activated Trop-2 is the Trop-2 molecule proteolytically cleaved between R87 and T88. The invention also involves the use of CAR constructs for use in anti-tumor cell therapy.

### STATE OF THE ART

Methods are available for adoptive cell therapy using engineered cells expressing recombinant receptors, such as chimeric antigen receptor (CARs) containing extracellular antibody antigen-binding domains.

CAR-T or -NK cell therapy is a promising T/NK cell therapeutic engineering practice. This is based on artificial CARs that target specific tumor-associated antigens and directly connect the antibody-antigen recognition with the cytotoxic activities of immune effector cells. Immune cells are isolated from the patient's peripheral blood (a procedure known as leukapheresis) to be engineered in vitro to express a specific CAR and once infused back in the patient they trigger an immune response against the corresponding target antigen and cells expressing that antigen.

Human defense system can identify molecules as self or non-self, including bacteria, viruses, and abnormal cancer cells. The identification of cancer cells is based on their antigenicity and immunogenicity acquired through the expression of antigens identified as non-self. However, cancer cells can subvert the immune system to their advantage, resulting in inadequate antitumor immunity and tumor survival and progression.

The generation of CARs has revolutionized T-cell-based immunotherapy for the treatment of certain cancers. CAR-T recognize the target antigen through immunoglobulin antigen-binding regions, hence by-passing the need for MHC (Major Histocompatibility Complex) presentation.

CARs typically comprise an antibody fragment, such as a scFv or Fab fragment, incorporated in a fusion protein that also comprises additional components, such as a CD3-ζ or CD28 transmembrane domain and selective T-cell activating moieties, including the endodomains of CD3-ζ, CD28, OX40, 4-IBB, Lek and/or ICOS (Sadelain, Brentjens et al. 2013).

CAR constructs have been used to direct the activity of natural killer (NK) cells, too (Hermanson and Kaufman 2015). NK cells are immune cells that identify target cells in the absence of MHC and antibodies. Thus, they can elicit rapid immune responses (Klingemann 2014), making them important for the destruction of cells which are mostly devoid of MHC class I.

Cancer cells that do not cause any inflammation tend to be treated as self by the immune system and do not efficiently stimulate a T cell response. NK cells produce several cytokines, including tumor necrosis factor α, interferon γ and IL-10 (Jiang, Zhang et al. 2014). The activation of NK cells leads to the gradual formation of immune effectors cells, such as dendritic cells, macrophages, and neutrophils, which consequently facilitate antigen-specific T and B cell responses.

NK cells do not require HLA matching; therefore, they can be used as allogeneic effector cells (Hermanson and Kaufman 2015). The lack of endogenous antigen specificity for targeting cells to be killed, allows NK cells, unlike T cells, to be easily re-directed by CARs. The cell-targeting scFv or Fab may be linked via a transmembrane domain to one or more intracellular signalling domains to effect lymphocyte activation. Signalling domains used with CAR-NK cells have included CD3-ζ, CD28, 4-IBB, DAPIO and OX40. NK cell lines, e.g., NK-92, NKG, YT, NK-YS, HANK-I, YTS, NKL, have also been used to this end.

A major concern with CAR-T/NK therapy is the danger of a “cytokine storm” associated with intense anti-tumor responses, as mediated by large numbers of activated T cells (Sadelain, Brentjens et al. 2013). This cytokine storm leads to fever, hypotension, hypoxia, neurologic dysfunction and multiorgan failure.

Thus, a fundamental need exists for improved design of CAR constructs, and CAR strategies, with better efficacy and decreased systemic toxicity, and for implementing parallel approaches to reduce the risk of a “cytokine storm” or other systemic toxicities.

Prevention of these severe reactions may help to mitigate on-target toxicity on non-tumor cells, where normal tissues expressing the target antigen are affected by toxicity due to the CAR-T or CAR-NK therapy. For example, severe transient inflammatory colitis was induced in all three cancer patients with metastatic colorectal cancer who had received CEA-targeting T cells (Parkhurst, Yang et al. 2011).

A critical need thus exists for controlling and managing CAR-T and CAR-NK cell toxicity, particularly when it reaches life-threatening levels. Different strategies have been tried to implement robust approaches for taming CAR-T and CAR-NK cell toxicity.

One of the oldest strategies for preventing these off-target effects was based on the introduction of suicide genes in CAR-T cells, which are being utilized in cancer immunotherapy (Jones, Lamb et al. 2014). Different genetically encoded molecules allow to induce selective death for gene-modified T cells, through control of metabolic pathways, dimerization induction. These were designed to lead to irreversible elimination of cells responsible for the uncontrolled toxicity and prevention of damage to normal tissues. Two of the currently validated suicide genes are herpes-simplex-thymidine-kinase (HSV-TK) and inducible-caspase-9 (iCasp9) (Jones, Lamb et al. 2014).

Additional strategies involve the regulation of the suicide gene activation, for eliminating CAR-T cells while preserving a portion of them still capable of antitumor activity, and the introduction of inhibitory CARs for sparing normal cells (Minagawa, Al-Obaidi et al. 2019).

FDA recently approved tocilizumab, an anti-interleukin-6-receptor antagonist, for the treatment of CAR T-cell-induced cytokine release syndrome, together with glucocorticoids administration in refractory cases (Santomasso, Nastoupil et al. 2021).

However, these approaches are not satisfactory. They are mainly based on the introduction of additional genes. Many of these novel molecules that are being introduced in, and produced by, the CAR-T cells often induce immune response by the host, which then damages CAR-T/NK cells.

Niels Kaj Jerne in 1971 modelled how the functional development of immune system was shaped during early ontogeny. In the thymus, lymphocytes producing antibodies (Abs) against endogenous molecules are suppressed, leading to the survival of/selection for only “anti-non-self” antigens, recognized through processes of random mutation and clonal selection. This mechanism determines self-tolerance and wide Abs diversity. Immunoglobulin genes are formed through V (D) J recombination, which joins the variable (V), diversity (D), and joining (J) germline genes. This leads each individual to generate a different, unique range of Ab pattern, depending both on diverse histocompatibility antigens on antigen-presenting cells and on random mutations involving V-genes.

The variable region of T-cell receptors (TCRs) and immunoglobulins contain complementarity-determining regions (CDR) with unique amino acid structure, that determine antigen specificity. The structure formed by each CDR is referred to as an idiotope (Kieber-Emmons, Monzavi-Karbassi et al. 2012). The collection of idiotopes on a single antibody molecule determines that antibody's idiotype (Id) (Jerne 2004) (FIG. 1).

Immunization with anti-idiotype (anti-Id) antibodies powered immunotherapy approaches against cancer. According to the network hypothesis, the Id-anti-Id interactions regulate the immune response of a host to a given antigen, by inducing specific immune responses similar to responses induced by nominal antigen (Ag). Moreover, Id-based immunotherapy does not depend on vaccination with the original Ag nor its fragments, excluding the possibility of parallel undesired side effects associated with conventional antigen vaccines. Also, the predictability of the fine specificities of induced immune responses to tumours is higher for anti-Id vaccines than for antigen vaccines.

The authors of the present invention designed and showed the advantage of implementing a totally specific CAR-T/NK cell damping approach, in the absence of any additional genetic modification. This was achieved by developing and utilizing anti-Trop-2 anti-Id antibodies, as described below.

### DESCRIPTION

The present disclosure relates to anti-idiotype antibodies (anti-Id Ab) that bind to or recognize anti-Trop-2 antibody moieties, in particular, anti-Trop-2 antibody moieties present in recombinant receptors, including CARs.

The disclosure further relates to uses of anti-Id Ab for specifically identifying, detecting or selecting cells expressing such recombinant receptors, such as anti-Trop-2 CAR T cells, or anti-Trop-2 CAR NK cells.

The disclosure further relates to uses of anti-Id Ab to specifically deplete such cells.

### DESCRIPTION OF THE DRAWINGS

FIG. 1: Schematic representation of the effects elicited by the anti-Id Ab according to the present invention in the presence of an anti-Trop-2 CAR-T cell. Idiotypic determinants are generated by the conformation of the amino acid sequences of the heavy and light-chain variable regions, specific for each antigen. The anti-Id Ab recognizes antigenic determinants in the variable region of an antibody, expressed on the extracellular portion of the CAR T cell.

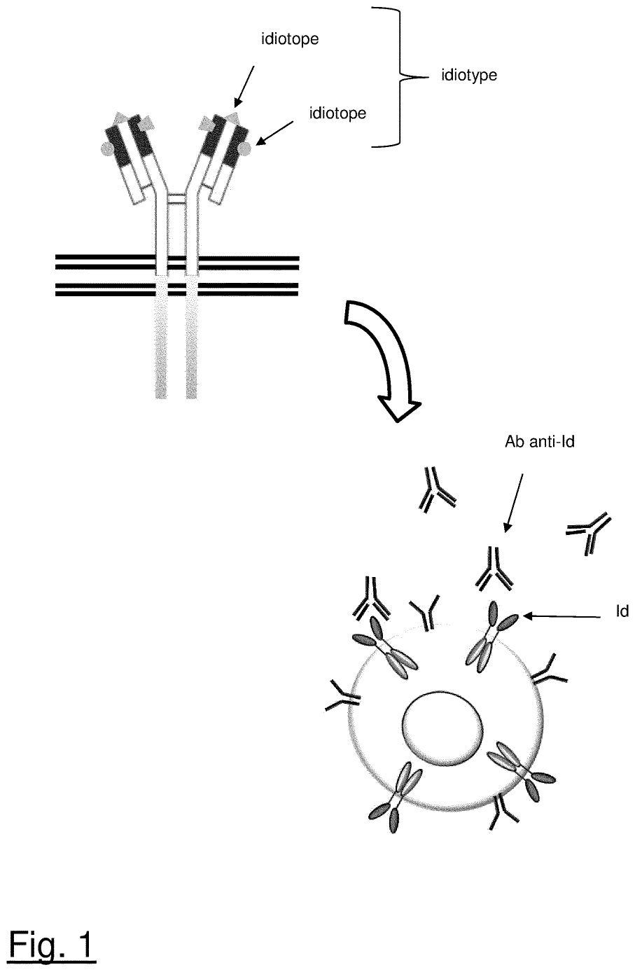

FIG. 2: Flow cytometry analysis of Jurkat cells transduced with anti-activated Trop-2 CAR. Horizontal axis: fluorescence staining. Vertical axis: cell forward scatter, related to cell dimensions. Each dot represents the reading for a single cell. (A) control profile of unstained cells. (B) anti-FLAG-Alexa488-antibody binding a synthetic tag, the Flag, added to the CAR construct. (C) anti-Hu2G10 idiotype (1D4-1 anti-Id Ab), followed by goat-anti-mouse-Alexa488 fluorescent antibodies to reveal binding. The ovals encompass positive CAR-expressing cells.

FIG. 3: ELISA assay demonstrating the affinity of the anti-Id Ab for the CAR variable regions. Wells of an ELISA plate were coated with the 1D4-1 anti-Id Ab and incubated with serial dilutions of the Hu2G10 mAb. Binding was detected using HRP-conjugated goat anti-human kappa polyclonal antibody. Absorbance values (Y-axis) are plotted at each antibody concentration tested (X-axis) in the figure. Background refers to a sample well in which all indicated procedures were performed, except no Hu2G10 was applied.

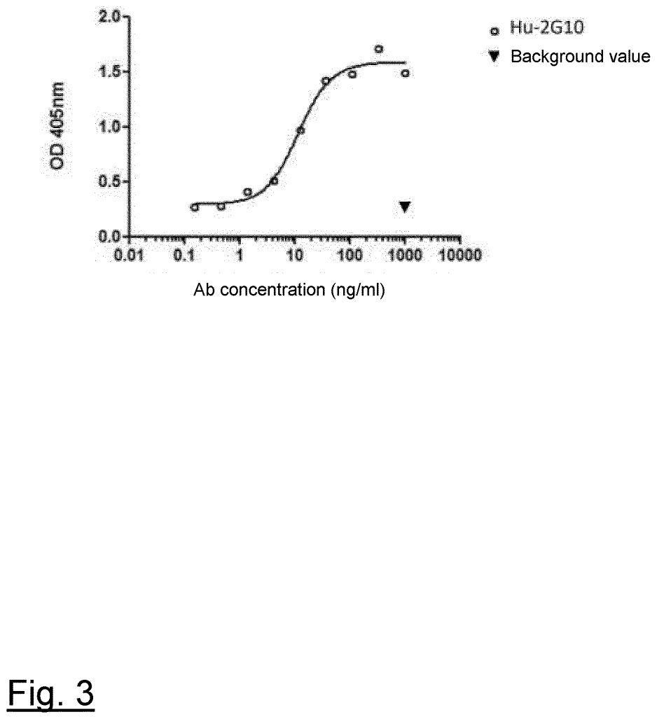

### DETAILED DESCRIPTION

Unless specifically defined herein, all technical and scientific terms used have the same meaning as commonly understood by a skilled person in the fields of gene therapy, biochemistry, genetics, and molecular biology.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Third Edition, (Sambrook et al, 2001, Cold Spring Harbor, New York: Cold Spring Harbor Laboratory Press).

Provided herein are agents that bind to or recognize antibodies and antigen-binding fragments thereof, including antibody fragments such as scFvs, and chimeric molecules containing the same, such as chimeric antigen receptors. Also provided are compositions and articles of manufacture containing such agents, including those including a surface to which the agent is bound, such as a solid surface, e.g., a plate or bead. Also, among the embodiments provided herein are uses and methods of using such agents, compositions, and articles, including for detection, use, manipulation and/or stimulation of cells or therapies containing or suspected of containing the antibody or chimeric molecule, such as in the detection, stimulation or use of CAR-expressing cells.

Trop-2, a hallmark of cancer cells and metastasis spreading, was chosen as target for CARs engineering, as extremely specific means for controlling CAR-T and CAR-NK activity and potential adverse effects, after administration for therapy. Trop-2-targeted analytical and therapeutic approaches known in the art have relied on anti-Trop-2 mAbs that recognize a single immunodominant epitope, poised between the globular and the stem regions, which is accessible and recognized in all Trop-2 expressing cells.

Provided herein, in some embodiments, is an anti-Id Ab or antigen-binding fragment thereof that binds to or recognizes an anti-Trop-2 target antibody or antigen-binding fragment thereof.

In an embodiment, said anti-Trop-2 antibody is 2G10 mAb, described in WO2010089782.

In an embodiment, said anti-Trop-2 antibody is humanized 2G10 mAb, described in WO2016087651.

In an embodiment, the target is activated Trop-2, i.e., a processed form of Trop-2 with specific and differential expression in tumor cells, wherein said processed form of Trop-2 is proteolytically cleaved between R87 and T88.

Anti-activated Trop-2 antibodies wherein activated Trop-2 is the Trop-2 molecule proteolytically cleaved between R87 and T88 were generated using an immunogen comprising both the entire extracellular portion (SEQ ID NO: 1, corresponding to amino acids 31-274 of SEQ ID NO: 2) and single domains of the Trop-2 molecule (SEQ ID NO: 3, globular domain: amino acids 31-145 of SEQ ID NO: 1; SEQ ID NO: 4 “stem”: amino acids 146-274 of SEQ ID NO: 2). These were produced in their native folding in human 293 transformed kidney epithelial cells and MCF-7 breast adenocarcinoma, and murine L fibrosarcoma and NS-0 myeloma, in insect Sf9 and yeast cells. Expression vectors for production were generated using PCR amplification of Trop-2 coding sequences fused to tags for purification or immunogenicity enhancement. The PCR fragments were subcloned in the vectors described above and expressed in the corresponding hosts. The Trop-2 proteins were purified by affinity chromatography. BALB/c mice were subjected to multiple immunization cycles with the immunogen described above, following best procedures known in the art. Splenocytes from immunized mice were fused to Sp2/0 or NS-0 myeloma cells and corresponding hybridomas were obtained, according to the methods known in the art. The antibodies produced by the hybridomas thus obtained were screened for specific and differential reactivity towards the processed Trop-2 that is expressed by tumor cells. mAbs 1A9 and 1B4 have been selected for their ability to recognize and bind with high affinity only the tumor-specific processed form of Trop-2 (activated Trop-2, wherein said activated Trop-2 is the Trop-2 molecule proteolytically cleaved between R87 and T88), and not the unprocessed Trop-2 found in normal tissues. Flow cytometry cross-competition experiments between 1A9, 1B4 and T16 mAbs on KM12SM/wtTrop-2 and KM12SM/vector transfectants demonstrated that 1A9 and 1B4 blocked each other's binding, thus indicating recognition of the same epitope. On the contrary, there is no competition of 1A9 and 1B4 against T16, demonstrating how the procedures here described have effectively allowed to obtain mAbs for an epitope that is different from the immunodominant one.

In an embodiment, said anti activated Trop-2 antibody is 1A9, secreted by the hybridoma deposited with the International Depositary Authority (IDA): Interlab Cell Line Collection, IRCCS Ospedale Policlinico San Martino, Genova, Italy, and assigned accession number PD21006.

In an embodiment, said anti activated Trop-2 antibody is 1B4, secreted by the hybridoma deposited with the International Depositary Authority (IDA): Interlab Cell Line Collection, IRCCS Ospedale Policlinico San Martino, Genova, Italy, and assigned accession number PD21005.

C57BL/6 have been immunized with humanized 2G10 antibody, then stimulated with a murine 2G10 antibody. Hybridomas have been obtained, a preferred one being 1D4-1 anti-Id Ab.

In a preferred embodiment, said 1D4-1 anti-Id Ab is secreted by the hybridoma deposited with the International Depositary Authority (IDA): Interlab Cell Line Collection, IRCCS Ospedale Policlinico San Martino, Genova, Italy, and assigned accession number PD21004.

In some of any such embodiments, the anti-Id Ab or antigen-binding fragment specifically binds the anti-Trop-2 target antibody or antigen-binding fragment comprised within or included in an antigen-binding domain of an extracellular portion of a CAR.

In some of any such embodiments, the scFv is within or included in an extracellular portion of a CAR. In some of any such embodiments, the anti-Id Ab or antigen-binding fragment binds the scFv comprised within or included in an extracellular portion of a CAR. In some of any such embodiments, the CAR further comprises a transmembrane domain linked to the antigen-binding domain via a spacer. In some of any such embodiments, the spacer is an immunoglobulin spacer. In some of any such embodiments, the spacer comprises the amino acid motif GlyGlyGlyGlySer, more preferably, said motif is repeated 4 times. In some of any such embodiments, the transmembrane domain comprises a transmembrane portion of CD28. In some of any such embodiments, the transmembrane portion of CD28 is human CD28. In some of any such embodiments, the anti-Id Ab or antigen-binding fragment thereof does not bind to an epitope in the spacer domain of the CAR.

In some of any such embodiments, the anti-Id Ab or antigen-binding fragment thereof does not bind to an epitope in an Fc domain. In some of any such embodiments, the Fc domain is a human IgG1 Fc domain.

In some of any such embodiments, the anti-Id Ab or antigen-binding fragment thereof is an agonist of a CAR comprising the anti-Trop-2 target antibody or antigen-binding fragment thereof. In some of any such embodiments, the anti-Id Ab or antigen-binding fragment thereof is an agonist of the CAR when in soluble form. In some of any such embodiments, the anti-Id Ab or antigen-binding fragment thereof is an agonist of the CAR when immobilized to a support or a stationary phase. In some of any such embodiments, the support or stationary phase is a plate or a bead.

In some of any such embodiments, the anti-Id Ab has a binding affinity (Kd) to the anti-Trop-2 target antibody or antigen-binding fragment thereof that is at or about or less than at or about 100 nM, 50 nM, 40 nM, 30 nM, 25 nM, 20 nM, 19 nM, 18 nM, 17 nM, 16 nM, 15 nM, 14 nM, 13 nM, 12 nM, 11 nM, 10 nM, 9 nM, 8 nM, 7 nM, 6 nM, 5 nM, 4 nM, 3 nM, 2 nM, or 1 nM or 0.1 nM or 0.01 nM or 0.001 nM.

In some of any such embodiments, the anti-Id Ab or antigen-binding fragment thereof is an intact antibody or full-length antibody.

Also provided herein, in some embodiments, is a conjugate, comprising the anti-Id Ab or antigen-binding fragment thereof of any of the embodiments provided herein and a heterologous molecule or moiety. In some of any such embodiments, the heterologous molecule or moiety is a label. In some of any such embodiments, the label is selected from a fluorescent dye, a fluorescent protein, a radioisotope, a chromophore, a metal ion, a gold particle, a silver particle, a magnetic particle, a polypeptide, an enzyme, a streptavidin, a biotin, a luminescent compound, and an oligonucleotide. In some of any such embodiments, the heterologous molecule or moiety is a protein, peptide, nucleic acid or small molecule, which optionally is or comprises a toxin or a

### Strep-Tag.

Also provided herein, in some embodiments, is a composition comprising the anti-Id Ab or antigen-binding fragment thereof of any one of such embodiments or the conjugate of any one of such embodiments.

In some of any such embodiments, the composition further comprises a pharmaceutically acceptable excipient.

In some of any such embodiments, the composition further comprises compounds active in cancer therapy.

Also provided herein, in some embodiments, is a kit, comprising one or more of the anti-Id Ab or antigen-binding fragments thereof of any one of such embodiments, the conjugate of any one of such embodiments, and, optionally, instructions for use.

Also provided herein, in some embodiments, is a method of detecting a CAR comprising a target antibody or antigen-binding fragment thereof, comprising:
(a) contacting a cell expressing a CAR comprising a target antibody or antigen-binding fragment thereof with the anti-Id Ab or antigen-binding fragment thereof of any one of such embodiments, or the conjugate of any one of such embodiments, that specifically binds to the target antibody or antigen-binding fragment thereof; and
(b) detecting cells bound with the anti-Id Ab or antigen-binding fragment thereof.

In some of any such embodiments, the anti-Id Ab or antigen-binding fragment thereof is directly or indirectly labelled for detection.

Also provided herein, in some embodiments, is a method of selecting cells from a cell population, comprising:
(a) contacting a cell population expressing a CAR comprising a target antibody or antigen-binding fragment thereof or a cell bound to a target antibody or antigen-binding fragment thereof with the anti-Id Ab or antigen-binding fragment thereof of any one of such embodiments, or the conjugate of any one of such embodiments, that specifically binds to the target antibody or antigen-binding fragment thereof; and
(b) selecting cells bound with the anti-Id Ab or antigen-binding fragment thereof. In some of any such embodiments, the cells bound with the anti-Id Ab or antigen-binding fragment thereof are selected by affinity-based separation.

In some of any such embodiments, the affinity-based separation is immunoaffinity-based separation. In some of any such embodiments, the affinity-based separation is by flow cytometry. In some of any such embodiments, the affinity-based separation is by magnetic activated cell sorting. In some of any such embodiments, the affinity-based separation comprises affinity chromatography. In some of any such embodiments, the anti-idiotype antibody or antigen-binding fragment thereof is reversibly bound or immobilized to a support or a stationary phase.

Also provided herein, in some embodiments, is a method of depleting cells, comprising administering, to a subject in need thereof, a composition comprising the anti-Id Ab or antigen-binding fragment thereof of any one of such embodiments, or the conjugate of any one of such embodiments, that specifically binds to a target antibody or antigen-binding fragment thereof, wherein the subject has been administered a cell expressing a CAR comprising the target antibody or antigen-binding fragment thereof. In some of any such embodiments, the depletion occurs via antibody-dependent cell-mediated cytotoxicity (ADCC).

In an embodiment, it is here claimed a pharmaceutical composition comprising an anti-Id Ab, preferably said anti-Id Ab is 1D4-1 Ab.

In an embodiment, said pharmaceutical composition further comprises a plurality of anti-Trop-2 CAR engineered cells.

In an embodiment, it is here claimed said pharmaceutical composition for use in a method for treating a disease in a subject. In an embodiment, said disease is a tumor.

In an embodiment, said anti-Id Ab tames the strength of the anti-Trop-2 CAR cells-based therapy.

The authors of the present invention surprisingly demonstrated that anti-Id Ab according to the present invention tames the toxicity of anti-Trop-2 CAR-T/NK cells.

Following are examples that illustrate procedures for practicing the invention. These examples should not be construed as limiting.

### EXAMPLES

### Example 1

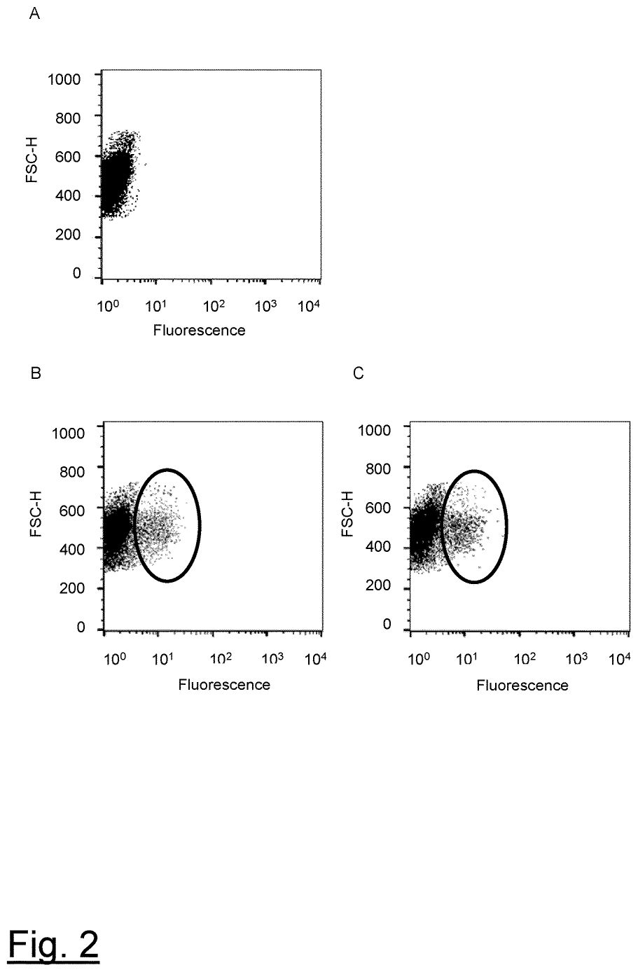

Jurkat cells were transduced with anti-activated Trop-2 CAR, wherein said VH has the sequence shown as SEQ ID NO: 5 for protein, SEQ ID NO: 6 for DNA and VL has the sequence shown as SEQ ID NO: 7 for protein, SEQ ID NO: 8 for DNA. Cells were then analyzed by FACS after staining with an anti-FLAG-Alexa488 antibody (FIG. 2B) or anti-Hu2G10 idiotype (1D4-1 anti-Id Ab) followed by goat-anti mouse-Alexa488 fluorescent antibodies (FIG. 2C). Unstained cells were also analyzed as control (FIG. 2A). The CAR elements showed fluorescence staining. i.e., CAR expression at the membrane level and capability to bind to the 1D4-1 anti-Id Ab.

SEQ ID NO: 5
QVQLVQSGAEVKKPGASVKVSCKASGFTFSSSYISWLRQAPGQRLEWI
AWIYAGTGGTSYNQKFTGKATLTVDTSASTAYMELSSLRSEDTAVYYC
ARHNPRYYAMDYWGQGTTVTVSS
SEQ ID NO: 6
CAAGTGCAGCTCGTCCAGTCTGGAGCTGAAGTCAAAAAGCCTGGGGCT
TCAGTGAAAGTCTCCTGCAAGGCTTCTGGCTTCACCTTCAGCAGTAGC
TATATCAGTTGGTTGAGGCAGGCCCCTGGACAGAGACTTGAGTGGATT
GCATGGATTTATGCTGGAACTGGCGGAACTAGCTATAATCAGAAGTTC
ACAGGCAAGGCCACACTGACTGTAGACACATCCGCCAGCACAGCCTAC
ATGGAACTCAGCAGCCTGAGATCTGAGGACACTGCCGTCTATTACTGT
GCAAGACATAACCCTCGTTACTATGCTATGGACTACTGGGGCCAAGGA
ACCACAGTCACCGTCTCCTCA
SEQ ID NO: 7
DTQMTQSPSSLSASVGDRVTITCITSTDIDDDMNWYQQKPGKAPKLLI
SEGNTLRPGVPSRFSGSGYGTDFTFTISSLQPEDIATYYCLQSDNLPY
TFGGGTKVEIKR
SEQ ID NO: 8
GACACCCAGATGACCCAGTCTCCAAGCTCCCTGTCCGCCAGCGTGGGA
GATAGAGTCACCATCACATGCATCACCAGCACTGATATTGATGATGAT
ATGAACTGGTACCAGCAGAAGCCAGGGAAAGCTCCTAAGCTCCTGATT
TCAGAAGGCAATACTCTGCGCCCTGGAGTCCCATCCCGATTCTCCGGC
AGTGGCTATGGAACAGATTTTACCTTTACAATTAGCTCCCTGCAGCCA
GAAGATATTGCAACCTACTACTGTTTGCAAAGTGATAACCTGCCCTAC
ACCTTCGGAGGGGGGACCAAAGTCGAAATCAAACGG

### Example 2

Wells of an ELISA plate are coated overnight at 4° C. with 100 μl/well of 1 μg/ml 1D4-1 Ab (lot Sep. 26, 2014) in PBS. After blocking with SuperBlock (Thermo Fisher Scientific), Hu2G10 diluted to 1 μg/ml in SuperBlock spiked with 2% human serum is applied, and serially 3-fold diluted in SuperBlock with 2% serum. Binding is detected using HRP-conjugated goat anti-human kappa polyclonal antibody in SuperBlock. Absorbance values (Y-axis) are computed at each antibody concentration tested (X-axis). Background is computed in a sample well in which all indicated procedures are performed, except no Hu2G10 is applied.

### REFERENCES

Hermanson, D. L. and D. S. Kaufman (2015). “Utilizing chimeric antigen receptors to direct natural killer cell activity.” Front Immunol 6:195.
Jerne, N. K. (2004). “The somatic generation of immune recognition. 1971.” Eur J Immunol 34 (5): 1234-1242.
Jiang, H., W. Zhang, P. Shang, H. Zhang, W. Fu, F. Ye, T. Zeng, H. Huang, X. Zhang, W. Sun, D. Man-Yuen Sze, Q. Yi and J. Hou (2014). “Transfection of chimeric anti-CD138 gene enhances natural killer cell activation and killing of multiple myeloma cells.” Mol Oncol 8 (2): 297-310.
Jones, B. S., L. S. Lamb, F. Goldman and A. Di Stasi (2014). “Improving the safety of cell therapy products by suicide gene transfer.” Front Pharmacol 5:254.
Kieber-Emmons, T., B. Monzavi-Karbassi, A. Pashov, S. Saha, R. Murali and H. Kohler (2012). “The promise of the anti-idiotype concept.” Front Oncol 2:196.
Klingemann, H. (2014). “Are natural killer cells superior CAR drivers?” Oncoimmunology 3: e28147.
Minagawa, K., M. Al-Obaidi and A. Di Stasi (2019). “Generation of Suicide Gene-Modified Chimeric Antigen Receptor-Redirected T-Cells for Cancer Immunotherapy.” Methods Mol Biol 1895:57-73.
Parkhurst, M. R., J. C. Yang, R. C. Langan, M. E. Dudley, D. A. Nathan, S. A. Feldman, J. L. Davis, R. A. Morgan, M. J. Merino, R. M. Sherry, M. S. Hughes, U. S. Kammula, G. Q. Phan, R. M. Lim, S. A. Wank, N. P. Restifo, P. F. Robbins, C. M. Laurencot and S. A. Rosenberg (2011). “T cells targeting carcinoembryonic antigen can mediate regression of metastatic colorectal cancer but induce severe transient colitis.” Mol Ther 19 (3): 620-626.
Sadelain, M., R. Brentjens and I. Riviere (2013). “The basic principles of chimeric antigen receptor design.” Cancer Discov 3 (4): 388-398.
Santomasso, B. D., L. J. Nastoupil, S. Adkins, C. Lacchetti, B. J. Schneider, M. Anadkat, M. B. Atkins, K. J. Brassil, J. M. Caterino, I. Chau, M. J. Davies, M. S. Ernstoff, L. Fecher, P. Funchain, I. Jaiyesimi, J. S. Mammen, J. Naidoo, A. Naing, T. Phillips, L. D. Porter, C. A. Reichner, C. Seigel, J.-M. Song, A. Spira, M. Suarez-Almazor, U. Swami, J. A. Thompson, P. Vikas, Y. Wang, J. S. Weber, K. Bollin and M. Ghosh (2021). “Management of Immune-Related Adverse Events in Patients Treated With Chimeric Antigen Receptor T-Cell Therapy: ASCO Guideline.” Journal of Clinical Oncology 39 (35): 3978-3992.

1. An anti-idiotype antibody (anti-Id Ab) or antigen-binding fragment thereof, wherein the anti-Id Ab or antigen-binding fragment thereof specifically binds the idiotype region of an anti-Trop-2 antibody or an antibody fragment of the anti-Trop-2 antibody.

2. The anti-Id Ab according to claim 1, which is isolated monoclonal 1D4-1 anti-Id Ab produced by hybridoma cell line deposited with the International Depositary Authority (IDA): Interlab Cell Line Collection, IRCCS Ospedale Policlinico San Martino, Genova, Italy, and assigned accession number PD21004.

3. The anti-Id Ab according to claim 1, wherein said anti-Trop-2 antibody or antibody fragment specifically recognises activated Trop-2, wherein said activated Trop-2 is the Trop-2 molecule proteolytically cleaved between R87 and T88.

4. The anti-Id Ab according to claim 1, binding the anti-Trop2 target antibody or antigen-binding fragment comprised within or included in an antigen-binding domain of an extracellular portion of a chimeric antigen receptor or CAR.

5. A pharmaceutical composition comprising the anti-Id Ab according to claim 1.

6. The composition according to claim 5, further comprising one or more additional compounds active in cancer therapy.

7. The composition according to claim 5, further comprising a plurality of anti-Trop-2 CAR engineered cells.

8. A method of detecting a CAR comprising a target antibody or antigen-binding fragment thereof, comprising:
(a) contacting a cell expressing a CAR comprising a target antibody or antigen-binding fragment thereof with the anti-Id Ab or antigen-binding fragment thereof according to claim 1, or the conjugate of any one of them, that specifically binds to the target antibody or antigen-binding fragment thereof; and
(b) detecting cells bound with the anti-Id Ab or antigen-binding fragment thereof.

9. A method of selecting cells expressing a CAR, comprising:
(a) contacting a cell population expressing a CAR comprising a target antibody or antigen-binding fragment thereof or a cell bound to a target antibody or antigen-binding fragment thereof with the anti-Id Ab or antigen-binding fragment thereof according to claim 1 or the conjugate that specifically binds to the target antibody or antigen-binding fragment thereof; and
(b) selecting cells bound with the anti-Id Ab or antigen-binding fragment thereof.

10. The method of detecting a CAR comprising a target antibody or antigen-binding fragment thereof according to claim 8, wherein, in said target antibody or antigen-binding fragment, VH has the sequence shown as SEQ ID NO: 5 for protein, SEQ ID NO: 6 for DNA and VL has the sequence shown as SEQ ID NO: 7 for protein, SEQ ID NO: 8 for DNA.

11. The method according to claim 10, wherein said anti-Id A is 1D4-1.

12. The method of selecting cells expressing a CAR according to claim 9, wherein, in said target antibody or antigen binding fragment, VH has the sequence shown as SEQ ID NO: 5 for protein, SEQ ID NO: 6 for DNA and VL has the sequence shown as SEQ ID NO: 7 for protein, SEQ ID NO: 8 for DNA.

13. The method according to claim 12, wherein said anti-Id A is 1D4-1.

14. The composition according to claim 6 formulated for use in a method for treating a disease in a subject.

15. The composition according to claim 14, wherein said disease is a solid tumour expressing TROP-2.

16. The composition according to claim 14, wherein the use of said composition comprises administration of a plurality of cells expressing a CAR comprising the target antibody or antigen-binding fragment thereof prior to or in combination with administration of said composition.

17. A method for treating a disease in a subject comprising administering the anti-idiotype antibody (anti-Id Ab) or antigen-binding fragment thereof of claim 1 to a subject in need thereof.

18. The method of claim 17, wherein the disease is cancer.
